(19) United States
(12) Patent Application Publication
(10) Pub. No.: US 2022/0241061 A1
(43) Pub. Date: Aug. 4, 2022

(54) ORAL DRUG DELIVERY SYSTEM AND METHOD

(71) Applicant: Universal Orthodontic Lab, Inc., Norwalk, CA (US)

(72) Inventors: Paul Kim, Diamond Bar, CA (US); Joseph Kim, Diamond Bar, CA (US)

(21) Appl. No.: 17/337,938

(22) Filed: Jun. 3, 2021

Related U.S. Application Data
(63) Continuation-in-part of application No. 17/163,057, filed on Jan. 29, 2021.

(51) Int. Cl. A61C 19/06; A61C 9/00; A61P 1/02

(52) U.S. Cl. CPC A61C 19/066; A61P 1/02; A61C 9/004

(57) ABSTRACT

The present invention provides an oral drug delivery system (“ODDS”) and methods of using and making the same. The ODDS comprises a tray fitted to at least some or all teeth of a patient's upper or lower arch for the application of medications to the patient in need of a medication. The tray is formed from a resilient elastomeric material molded to substantially conform to the teeth and gum tissue, the tray having at least one cavity formed to conform to the teeth, one or more raised ridges surrounding the cavity corresponding to a location along the patient's gum line, and one or more negative grooves configured to receive a medication by the raised ridges, and the negative grooves surrounding the cavity corresponding to an intersection of the teeth and gum line. A tooth structure medication can be included in the cavity apart from the one or more negative grooves.

### CROSS-REFERENCE TO RELATED APPLICATIONS

This is a continuation-in-part of U.S. application Ser. No. 17/163,057, filed on Jan. 29, 2021, the teaching of which is incorporated herein by reference in its entirety.

### TECHNICAL FIELD OF THE INVENTION

This invention generally relates to an oral drug delivery system and methods of making and using the same.

### BACKGROUND OF THE INVENTION

Background information related to the present disclosure as described herein may not constitute prior art.

It is known that gingival cavity is the growth bed of a plethora of bacteria, and medical research has demonstrated a significant source of systemic disease is related to specific gingival bacteria, with special emphasis on certain gram negative anaerobic bacteria that have been found associated with a variety of systemic inflammatory responses and appear to originate only in periodontal pockets and enter the bloodstream through a close proximity to the host circulatory system. Additionally, it has been found that these gingival periopathogens can become systemic. Research has demonstrated that bacteremia was observed in 100% of the patients after dental extraction and in 70% after dental scaling. Mastication on infected gum tissues increases systemic bacterial endotoxins levels four-fold. Simple gingival hygiene procedures, such as brushing of the teeth, can increase the prevalence of bacteremia from 17 to 40%. Research has also demonstrated that the dissemination of gingival microorganisms into the bloodstream is common. In fact, it has been found that within less than 1 minute after a gingival procedure, organisms from an infected site can reach the heart, lungs, and peripheral blood capillary system of a patient. These periopathogens can cause host injury (exotoxins and toxic bacterial products), inflammation (immune system-antigen/antibody reactions) and infections (bacteremia) in a person.

Meanwhile, while IV and subcutaneous injection are effective delivery methods, such delivery methods can be unavailable or not applicable to patients of needle aversion or of a medical condition where injection is not suitable. As such, there is a continued need to explore and make use of delivery routes that can by-pass the gastric such that a drug molecule, e.g., a peptide drug, can minimize damage to its structure.

On the other hand, drug delivery through the mouth for a variety of gingival or systemic diseases is gaining popularity as an alternative to traditional medicament delivery due to its efficacy, comfort, and non-invasive nature. The disclosed drug delivery system and method of manufacture thereof for treating a variety of gingival or systemic diseases include an apparatus having a body formed to be fitted on and around the upper or lower teeth composed of a flexible thermoforming plastic material that substantially conforms to the teeth and gum tissue surrounding the teeth, with a raised ridge that provides positive pressure and a seal, and a grooved reservoir that holds the medicine in place thereby minimizing leakage.

Therefore, there is a continuous need for drug delivery system that addresses the above-identified issues or problems. The embodiments described below address such issues or problems.

### SUMMARY OF THE INVENTION

In one aspect of the present invention, it is provided an oral drug delivery system (“ODDS”), comprising:

a tray fitted to at least some or all teeth of a patient's upper or lower arch for the application of medications to the patient in need of a medication, the tray being of resilient elastomeric material molded to substantially conform to the teeth and gum tissue, the tray having at least one cavity formed to conform to the teeth,
one or more raised ridges surrounding the cavity corresponding to a location along the patient's gum line,
one or more negative grooves configured to receive a medication by the raised ridges, the negative grooves surrounding the cavity corresponding to an intersection of the teeth and gum line,
wherein the raised ridge creates a positive pressure on the gum and a seal, thereby minimizing leakage of medicine, and
wherein, as compared with the one or more negative grooves, the one or more raised ridges are further away from the intersection of the teeth and gum line.

In some embodiments of the invention ODDS, optionally in combination with any of the invention embodiments disclosed herein, the one or more negative grooves are filled with a medication forming a drug reservoir.

In some embodiments of the invention ODDS, optionally in combination with any of the invention embodiments disclosed herein, the medication comprises a pharmaceutically active agent.

In some embodiments of the invention ODDS, optionally in combination with any of the invention embodiments disclosed herein, the pharmaceutically active agent is a small molecule drug.

In some embodiments of the invention ODDS, optionally in combination with any of the invention embodiments disclosed herein, the pharmaceutically active agent is a peptide or polypeptide drug.

In some embodiments of the invention ODDS, optionally in combination with any of the invention embodiments disclosed herein, the pharmaceutically active agent is an antimicrobial agent, an anti-depressant, an anti-neurodegenerative drug, an anti-inflammatory agent, or an anti-pain drug.

In some embodiments of the invention ODDS, optionally in combination with any of the invention embodiments disclosed herein, the at least one cavity apart from the negative grooves is filled with a tooth structure medication.

In some embodiments of the invention ODDS, optionally in combination with any of the invention embodiments disclosed herein, the tooth structure medication selected from one or more of a tooth whitening agent, tooth remineralizing agents, and a combination thereof.

In some embodiments of the invention ODDS, optionally in combination with any of the invention embodiments disclosed herein, the medication further comprises a pharmaceutically acceptable carrier.

In some embodiments of the invention ODDS, optionally in combination with any of the invention embodiments disclosed herein, the medication is in a solid formulation or in a solid formulation.

In another aspect of the present invention, it is provided a method of forming an ODDS (“ODDS”), comprising:

forming a female impression of a patient's teeth and gums that support the teeth from a suitable hardenable material or performing a digitized scan of a patient's teeth and gums that support the teeth,

forming a digitized male models of the patient's teeth and gums,

programming the digitized male model to incorporate one or more raised ridges along an intersection of the teeth and gum line and one or more depressed grooves by the raised ridges along the intersection of the teeth and gum line, the raised ridges being closer to the intersection of the teeth and gum line as compared with that of the one or more depressed grooves,

forming a male model from the digitized male model,

forming a tray by molding a resilient material over the male model, thereby generating the ODDS having the tray,

wherein the tray is fitted to at least some or all teeth of a patient's upper or lower arch for the application of medications to the patient in need of a medication, the tray being of resilient elastomeric material molded to substantially conform to the teeth and gum tissue, and the tray having
at least one cavity formed to conform to the teeth,
one or more raised ridges surrounding the cavity corresponding to a location along the patient's gum line,
one or more negative grooves configured to receive a medication by the raised ridges, the negative grooves surrounding the cavity corresponding to an intersection of the teeth and gum line,
wherein the raised ridge creates a positive pressure on the gum and a seal, thereby minimizing leakage of medicine, and
wherein, as compared with the one or more negative grooves, the one or more raised ridges are further away from the intersection of the teeth and gum line.

In some embodiments of the invention method, optionally in combination with any of the invention embodiments disclosed herein, forming a male model from the digitized male model is by 3D printing.

In some embodiments of the invention method, optionally in combination with any of the invention embodiments disclosed herein, the method further comprising filling a medication to the one or more negative grooves to form a drug reservoir.

In some embodiments of the invention method, optionally in combination with any of the invention embodiments disclosed herein, the medication comprises a pharmaceutically active agent.

In some embodiments of the invention method, optionally in combination with any of the invention embodiments disclosed herein, the pharmaceutically active agent is a small molecule drug or a peptide or polypeptide drug.

In some embodiments of the invention method, optionally in combination with any of the invention embodiments disclosed herein, the pharmaceutically active agent is an antimicrobial agent, an anti-depressant, an anti-neurodegenerative drug, an anti-inflammatory agent, or an anti-pain drug.

In some embodiments of the invention method, optionally in combination with any of the invention embodiments disclosed herein, further comprising filling the at least one cavity apart from the one or more grooves with a tooth structure medication.

In some embodiments of the invention method, optionally in combination with any of the invention embodiments disclosed herein, the tooth structure medication is selected from the group consisting of a tooth whitening agent, tooth remineralizing agents, and a combination thereof.

In some embodiments of the invention method, optionally in combination with any of the invention embodiments disclosed herein, the medication further comprises a pharmaceutically acceptable carrier.

In some embodiments of the invention ODDS, optionally in combination with any of the invention embodiments disclosed herein, the medication is in a solid formulation or in a solid formulation.

In another aspect of the present invention, it is provided a method of treating a medical condition in a patient, comprising:

determining a presence of a medical condition in the patient,

forming an ODDS (“ODDS”), the ODDS comprising
a tray fitted to at least some or all teeth of a patient's upper or lower arch for the application of medications to the patient in need of a medication, the tray being of resilient elastomeric material molded to substantially conform to the teeth and gum tissue, the tray having at least one cavity formed to conform to the teeth;
one or more raised ridges surrounding the cavity corresponding to a location along the patient's gum line;
one or more negative grooves as a drug reservoir filled with a medication by the raised ridges, the negative grooves surrounding the cavity corresponding to an intersection of the teeth and gum line; and
optionally a tooth structure medication included in the cavity apart from the negative grooves,
wherein the raised ridge creates a positive pressure on the gum and a seal, thereby minimizing leakage of medicine; and
wherein, as compared with the one or more negative grooves, the one or more raised ridges are further away from the intersection of the teeth and gum line, and

applying the ODDS to the patient.

In some embodiments of the invention method, optionally in combination with any of the invention embodiments disclosed herein, the medication comprises a pharmaceutically active agent that is a small molecule drug or a peptide or polypeptide drug.

In some embodiments of the invention method, optionally in combination with any of the invention embodiments disclosed herein, the medication comprises a pharmaceutically active agent that is an antimicrobial agent, an anti-depressant, an anti-neurodegenerative drug, an anti-inflammatory agent, or an anti-pain drug.

In some embodiments of the invention method, optionally in combination with any of the invention embodiments disclosed herein, the medication further comprises a pharmaceutically acceptable carrier.

In some embodiments of the invention method, optionally in combination with any of the invention embodiments disclosed herein, the medication is in a solid formulation or in a solid formulation.

In some embodiments of the invention method, optionally in combination with any of the invention embodiments disclosed herein, the medical condition is a periodontal disease.

In some embodiments of the invention method, optionally in combination with any of the invention embodiments disclosed herein, the tooth structure medication is one of a tooth whitening agent, tooth remineralizing agents, and a combination thereof.

### BRIEF DESCRIPTION OF THE DRAWINGS

FIG. 1 shows a buccal view of tray 1 of an embodiment of the oral drug delivery system (“ODDS”) of invention.

FIG. 2 is a gingival view of tray 1 of an embodiment of the ODDS of invention.

FIG. 3 is an occlusal view of tray 1 of an embodiment of the ODDS of invention.

FIG. 4 is a mold lb for forming tray 1.

FIG. 5 is an occlusal view of mold 1b for forming tray 1 of an embodiment of the ODDS of invention.

FIG. 6 shows a tray 1 formed on a mold 1b of an embodiment of the ODDS of invention.

### DETAILED DESCRIPTION OF THE INVENTION

Definitions

As used herein, the term “hardenable material” refers to a material used in dentistry to create a female impression of a dental arch or one used to create a male model of a dental arch.

As used herein, the term “resilient elastic material” refers to a polymeric material capable of forming a tray fitted to and conforming to each tooth of a dental arch and maintaining close contact with gum surface so as to form a close seal of the interior surface with respect to gum surface along the gum line of a tooth. In this context, a “positive pressure” is generated when a tray disclosed herein formed from the “resilient elastic material” is applied to a patient and a force is applied onto the tray, e.g., a biting by the patient or a tongue touch on the tray. Such “positive pressure” is generally temporary and as such, on-and-off biting and tongue touch would generative a high/low pressure cycles with the low point being the ambient pressure of the oral cavity so as to facilitate gingival drug delivery.

As used herein, the term “pharmaceutically active agent” can be any drug or agent that exert statistically significant beneficial pharmacological effect on a patient as compared with a scenario where no such agent drug or agent is used. Such statistically significant beneficial pharmacological effect can be a therapeutically significant effect. In this context, the term “therapeutically effective amount” and similar terms, if used herein, refers to an amount of a compound or combination of compounds that shows a pharmacological effect when administered in the mammalian body, such as ameliorates, attenuates or eliminates one or more symptoms of a particular disease or condition or prevents or delays the onset of one or more symptoms or a particular disease or condition.

The terms “treating,” “treat,” “treatment,” as used herein, include curative, preventative (e.g., prophylactic) and palliative treatment.

The term “biomaterial”, if used, includes scaffolds, hydro-gels, synthetic, artificial or natural materials which are bio-compatible for use in a mammalian medical/surgical context.

The terms “controlled release,” “sustained release,” and similar terms, if used herein, refer to the delivery of a com-pound or combination of compounds that ameliorates, attenu-ates or eliminates one or more symptoms of a particular disease or condition or prevents or delays the onset of one or more symptoms or a particular disease or condition over a predetermined period of time at a constant or variable rate, preferably a relatively constant rate, that maintains a concen-tration of active ingredient equivalent to a therapeutically effective amount over substantially all of the predetermined period of time.

As used herein, the term “patient” shall mean any mammalian subject, which can be human subject or an animal subject.

Oral Drug Delivery System

Accordingly, In one aspect of the present invention, it is provided an oral drug delivery system (“ODDS”), comprising:

a tray fitted to at least some or all teeth of a patient's upper or lower arch for the application of medications to the patient in need of a medication, the tray being of resilient elastomeric material molded to substantially conform to the teeth and gum tissue, the tray having at least one cavity formed to conform to the teeth,

one or more raised ridges surrounding the cavity corresponding to a location along the patient's gum line,

one or more negative grooves configured to receive a medication by the raised ridges, the negative grooves surrounding the cavity corresponding to an intersection of the teeth and gum line,

wherein the raised ridge creates a positive pressure on the gum and a seal, thereby minimizing leakage of medicine, and

wherein, as compared with the one or more negative grooves, the one or more raised ridges are further away from the intersection of the teeth and gum line.

In some embodiments of the invention ODDS, optionally in combination with any of the invention embodiments disclosed herein, the one or more negative grooves are filled with a medication forming a drug reservoir.

In some embodiments of the invention ODDS, optionally in combination with any of the invention embodiments disclosed herein, the medication comprises a pharmaceutically active agent.

In some embodiments of the invention ODDS, optionally in combination with any of the invention embodiments disclosed herein, the pharmaceutically active agent is a small molecule drug.

In some embodiments of the invention ODDS, optionally in combination with any of the invention embodiments disclosed herein, the pharmaceutically active agent is a peptide or polypeptide drug.

In some embodiments of the invention ODDS, optionally in combination with any of the invention embodiments disclosed herein, the pharmaceutically active agent is an antimicrobial agent, an anti-depressant, an anti-neurodegenerative drug, an anti-inflammatory agent, or an anti-pain drug.

In some embodiments of the invention ODDS, optionally in combination with any of the invention embodiments disclosed herein, the at least one cavity apart from the negative grooves is filled with a tooth structure medication.

In some embodiments of the invention ODDS, optionally in combination with any of the invention embodiments disclosed herein, the tooth structure medication selected from one or more of a tooth whitening agent, tooth remineralizing agents, and a combination thereof.

In some embodiments of the invention ODDS, optionally in combination with any of the invention embodiments disclosed herein, the medication further comprises a pharmaceutically acceptable carrier.

In some embodiments of the invention ODDS, optionally in combination with any of the invention embodiments disclosed herein, the medication is in a solid formulation or in a solid formulation.

Methods of Fabrication

In another aspect of the present invention, it is provided a method of forming an ODDS (“ODDS”), comprising:

forming a female impression of a patient's teeth and gums that support the teeth from a suitable hardenable material or performing a digitized scan of a patient's teeth and gums that support the teeth,

forming a digitized male models of the patient's teeth and gums,

programming the digitized male model to incorporate one or more raised ridges along an intersection of the teeth and gum line and one or more depressed grooves by the raised ridges along the intersection of the teeth and gum line, the raised ridges being closer to the intersection of the teeth and gum line as compared with that of the one or more depressed grooves,

forming a male model from the digitized male model,

forming a tray by molding a resilient material over the male model, thereby generating the ODDS having the tray,

wherein the tray is fitted to at least some or all teeth of a patient's upper or lower arch for the application of medications to the patient in need of a medication, the tray being of resilient elastomeric material molded to substantially conform to the teeth and gum tissue, and the tray having
at least one cavity formed to conform to the teeth,
one or more raised ridges surrounding the cavity corresponding to a location along the patient's gum line,
one or more negative grooves configured to receive a medication by the raised ridges, the negative grooves surrounding the cavity corresponding to an intersection of the teeth and gum line,
wherein the raised ridge creates a positive pressure on the gum and a seal, thereby minimizing leakage of medicine, and
wherein, as compared with the one or more negative grooves, the one or more raised ridges are further away from the intersection of the teeth and gum line.

In some embodiments of the invention method, optionally in combination with any of the invention embodiments disclosed herein, forming a male model from the digitized male model is by 3D printing.

In some embodiments of the invention method, optionally in combination with any of the invention embodiments disclosed herein, the method further comprising filling a medication to the one or more negative grooves to form a drug reservoir.

In some embodiments of the invention method, optionally in combination with any of the invention embodiments disclosed herein, the medication comprises a pharmaceutically active agent.

In some embodiments of the invention method, optionally in combination with any of the invention embodiments disclosed herein, the pharmaceutically active agent is a small molecule drug or a peptide or polypeptide drug.

In some embodiments of the invention method, optionally in combination with any of the invention embodiments disclosed herein, the pharmaceutically active agent is an antimicrobial agent, an anti-depressant, an anti-neurodegenerative drug, an anti-inflammatory agent, or an anti-pain drug.

In some embodiments of the invention method, optionally in combination with any of the invention embodiments disclosed herein, further comprising filling the at least one cavity apart from the one or more grooves with a tooth structure medication.

In some embodiments of the invention method, optionally in combination with any of the invention embodiments disclosed herein, the tooth structure medication is selected from the group consisting of a tooth whitening agent, tooth remineralizing agents, and a combination thereof.

In some embodiments of the invention method, optionally in combination with any of the invention embodiments disclosed herein, the medication further comprises a pharmaceutically acceptable carrier.

In some embodiments of the invention ODDS, optionally in combination with any of the invention embodiments disclosed herein, the medication is in a solid formulation or in a solid formulation.

Method of Use

In another aspect of the present invention, it is provided a method of treating a medical condition in a patient, comprising:

determining a presence of a medical condition in the patient,

forming an ODDS (“ODDS”), the ODDS comprising
a tray fitted to at least some or all teeth of a patient's upper or lower arch for the application of medications to the patient in need of a medication, the tray being of resilient elastomeric material molded to substantially conform to the teeth and gum tissue, the tray having at least one cavity formed to conform to the teeth;
one or more raised ridges surrounding the cavity corresponding to a location along the patient's gum line;
one or more negative grooves as a drug reservoir filled with a medication by the raised ridges, the negative grooves surrounding the cavity corresponding to an intersection of the teeth and gum line; and
optionally a tooth structure medication included in the cavity apart from the negative grooves,
wherein the raised ridge creates a positive pressure on the gum and a seal, thereby minimizing leakage of medicine; and wherein, as compared with the one or more negative grooves, the one or more raised ridges are further away from the intersection of the teeth and gum line, and

applying the ODDS to the patient.

In some embodiments of the invention method, optionally in combination with any of the invention embodiments disclosed herein, the medication comprises a pharmaceutically active agent that is a small molecule drug or a peptide or polypeptide drug.

In some embodiments of the invention method, optionally in combination with any of the invention embodiments disclosed herein, the medication comprises a pharmaceutically active agent that is an antimicrobial agent, an anti-depressant, an anti-neurodegenerative drug, an anti-inflammatory agent, or an anti-pain drug.

In some embodiments of the invention method, optionally in combination with any of the invention embodiments disclosed herein, the medication further comprises a pharmaceutically acceptable carrier.

In some embodiments of the invention method, optionally in combination with any of the invention embodiments disclosed herein, the medication is in a solid formulation or in a solid formulation.

In some embodiments of the invention method, optionally in combination with any of the invention embodiments disclosed herein, the medical condition is a periodontal disease.

In some embodiments of the invention method, optionally in combination with any of the invention embodiments disclosed herein, the tooth structure medication is one of a tooth whitening agent, tooth remineralizing agents, and a combination thereof.

Pharmaceutically Active Agent

While the benefits of the invention have been described with respect to certain drugs, some or all of these benefits are obtained when the formulation of the invention is used with a wide variety of drugs, such as immunosuppressants, antioxidants, anesthetics, chemotherapeutic agents, steroids (including retinoids), hormones, antibiotics, antivirals, antifungals, antiproliferatives, antihistamines, anticoagulants, antiphotoaging agents, melanotropic peptides, nonsteroidal and steroidal anti-inflammatory compounds, antipsychotics, and radiation absorbers, including UV-absorbers, chemotherapeutic agents, anti-nausea medication, and the like. Non-limiting examples of pharmacological materials or drugs suitable for use in the invention include anti-infectives (e.g., anti-bacterial agents) such as nitrofurazone, sodium propionate, antibiotics, including penicillin, tetracycline, oxytetracycline, chlorotetracycline, bacitracin, nystatin, streptomycin, neomycin, polymyxin, gramicidin, chloramphenicol, erythromycin, and azithromycin; sulfonamides, including sulfacetamide, sulfamethizole, sulfamethazine, sulfadiazine, sulfamerazine, peroxides (e.g., hydrogen peroxide or benzyl peroxide) and sulfisoxazole, and anti-virals including idoxuridine; antiallergenics such as antazoline, methapyritene, chlorpheniramine, pyrilamineprophenpyridamine, hydrocortisone, cortisone, hydrocortisone acetate, dexamethasone, dexamethasone 21-phosphate, fluocinolone, triamcinolone, medrysone, prednisolone, prednisolone 21-sodium succinate, and prednisolone acetate; desensitizing agents such as ragweed pollen antigens, hay fever pollen antigens, dust antigen and milk antigen; vaccines such as smallpox, yellow fever, distemper, hog cholera, chicken pox, antivenom, scarlet fever, dyptheria toxoid, tetanus toxoid, pigeon pox, whooping cough, influenzae rabies, mumps, measles, poliomyelitic, and Newcastle disease; decongestants such as phenylephrine, naphazoline, and tetrahydrazoline; miotics and anticholinesterases such as pilocarpine, esperine salicylate, carbachol, diisopropylfluorophosphate, phospholine iodide, and demecarium bromide; parasympatholytics such as atropine sulfate, cyclopentolate, homatropine, scopolamine, tropicamide, eucatropine, and hydroxyamphetamine; sympathomimetics such as epinephrine; sedatives and hypnotics such as pentobarbital sodium, phenobarbital, secobarbital sodium, codeine, (a-bromoisovaleryl) urea, carbromal; psychic energizers such as 3-(2-aminopropyl) indole acetate and 3-(2-aminobutyl) indole acetate; tranquilizers such as reserpine, chlorpromayline, and thiopropazate; androgenic steroids such as methyl-testosterone and fluorymesterone; estrogens such as estrone, 17-.beta.-estradiol, ethinyl estradiol, and diethyl stilbestrol; progestational agents such as progesterone, megestrol, melengestrol, chlormadinone, ethisterone, norethynodrel, 19-norprogesterone, norethindrone, medroxyprogesterone and 17-.beta.-hydroxy-progesterone; humgingival agents such as the prostaglandins, for example PGE1, PGE2 and PGF2; antipyretics such as aspirin, sodium salicylate, and salicylamide; antispasmodics such as atropine, methantheline, papaverine, and methscopolamine bromide; antimalarials such as the 4-aminoquinolines, 8-aminoquinolines, chloroquine, and pyrimethamine, antihistamines such as diphenhydramine, dimenhydrinate, tripelennamine, perphenazine, and chlorphenazine; cardioactive agents such as dibenzhydroflume thiazide, flumethiazide, chlorothiazide, and aminotrate; nutritional agents such as vitamins, natural and synthetic bioactive peptides and proteins, including growth factors, cell adhesion factors, cytokines, and biological response modifiers.

The dosage forms of a medication of the ODDS of the invention can be used to deliver any type of biologically active compound. Examples of such biologically active compounds delivered using the invention include opioids, CNS depressants and stimulants, as well as proteins, hormones, chemotherapeutic agents, anti-nausea medication, antibiotics, antivirals and other agents. One class of drug of particular interest for delivery using the system disclosed herein is opioids, which includes alfentanil, allylprodine, alphaprodine, anileridine, apomorphine, apocodeine, benzylmorphine, bezitramide, buprenorphine, butorphanol, clonitazene, codeine, cyclazocine, cyclorphen, cyprenorphine, desomorphine, dextromoramide, dezocine, diampromide, dihydrocodeine, dihydromorphine, dimenoxadol, dimepheptanol, dimethylthiambutene, dioxyaphetyl butyrate, dipipanone, eptazocine, ethoheptazine, ethylmethylthiambutene, ethylmorphine, etonitazene, fentanyl, heroin, hydrocodone, hydroxymethylmorphinan, hydromorphone, hydroxypethidine, isomethadone, ketobemidone, levallorphan, levorphanol, levophenacylmorphan, lofentanil, meperidine, meptazinol, metazocine, methadone, methylmorphine, metopon, morphine, myrophine, nalbuphine, narceine, nicomorphine, norlevorphanol, normethadone, nalorphine, normorphine, norpipanone, ohmefentanyl, opium, oxycodone, oxymorphone, papaveretum, pentazocine, phenadoxone, phenomorphan, phenazocine, phenoperidine, pholcodine, piminodine, piritramide, propheptazine, promedol, profadol, properidine, propiram, propoxyphene, remifentanyl, sufentanyl, tramadol, tilidine, naltrexone, naloxone, nalmefene, methylnaltrexone, naloxone methiodide, nalorphine, naloxonazine, nalide, nalmexone, nalbuphine, nalorphinedinicotinate, naltrindole (NTI), naltrindoleisothiocyanate, (NTH), naltriben (NTB), nor-binaltorphimine (nor-BNI), beta-funaltrexamine (b-FNA), BNTX, cyprodime, ICI-174,864, LY117413, MR2266, etorphine, DAMGO, CTOP, diprenorphine, naloxone benzoylhydrazone, bremazocine, ethylketocyclazocine, U50,488, U69,593, spiradoline, DPDPE, [D-Ala2,Glu4] deltorphin, DSLET, Met-enkephalin, Leu-enkephalin, 0-endorphin, dynorphin A, dynorphin B, a-neoendorphin, or an opioid having the same pentacyclic nucleus as nalmefene, naltrexone, buprenorphine, levorphanol, meptazinol, pentazocine, dezocine, or their pharmacologically effective esters or salts.

The dosage form disclosed allows for the release of drug including over a prolonged period, such as of several hours. The total period for release of drug in an amount sufficient to be an effective dosage may be greater than 20 hours, or greater than 17 hours, or greater than 15 hours, or greater than 12 hours, or greater than 10 hours, or greater than 8 hours, or greater than 6 hours, or greater than 4 hours, or greater than 2 hours, or greater than 1 hour. The amount of drug sufficient to provide an effective dosage is determined from the therapeutic range of the drug, which is determined from, for example, clinical trials, and this information is easily available to one of skill in the art. An example of dosage form for gingival delivery of various agents/drugs are described in U.S. Patent No. 9,918,982B2, the teaching of which is incorporated herein by reference.

Formulation and Carrier

The ODDS disclosed may include various components in addition to the carrier material (for example a HVLCM, described below in more detail). The additional compounds may be present in amounts ranging from about 75 wt % to as low as about 0.01 wt % of the total formulation. These additional components may include the following types of compounds:

Solvents, e.g., ethyl lactate (EL) or triacetine, DMSO, Propylene carbonate, NMP, Ethyl alcohol, Benzyl alcohol, Glycofurol, alpha-tocoperol, Miglyol 810, isopropyl alcohol, diethyl phthalate, PEG 400, triethyl citrate, benzyl benzoate.

Network formers, e.g., cellulose acetate butyrate (CAB 171-15, CAB 381-2 and CAB 381-20, supplied by Eastman Chemicals, the characteristics of which are described in Table 2); carbohydrate polymers, organic acids of carbohydrate polymers and other polymers, hydrogels, as well as particles such as silicon dioxide, ion exchange resins, and/or fiberglass, that are capable of associating, aligning or congealing to form three dimensional networks in an aqueous environment. Other examples include cellulose acetate phthalate, ethyl cellulose, Pluronic, Eudragit, Carbomer, hydroxyl propyl methyl cellulose, cellulose acetates such as CA 381-2 and cellulose triacetate, PMMA, CAB 500-5.

Rheology modifiers, e.g., caprylic/capric triglyceride (Migliol 810), isopropyl myristate (IM or IPM), ethyl oleate, triethyl citrate, dimethyl phthalate, and benzyl benzoate.

Stabilizers, e.g., antioxidants such as sodium citrate ascorbyl palmitate, and propyl gallate and/or reducing agents. Other examples include ascorbic acid, vitamin E, sodium bisulfate, butylhydroxyl toluene, BHA, acetylcysteine, monothioglycerol, phenyl-alpha-napthylamine, lecithin, EDTA.

In some embodiments, the ODDS disclosed herein may have a medication formulated to produce particular controlled plasma levels of drug over a particular period. This is obviously of great importance in maintaining a drug-plasma level within an appropriate therapeutic range. An appropriate therapeutic range will vary depending on the drug, but can range from femtogram/ml levels up to above microgram/mil levels for a desired period of time. For example, a single dose of a drug dosage form disclosed herein may result in maintenance of plasma levels of a drug at greater than 5 ng/ml for a period of greater than 8 hours. In other embodiments, the drug plasma level achieved using a single dose may be greater than 5 ng/ml for a period of greater than 10 hours, greater than 12 hours, greater than 14 hours, greater than 16 hours, greater than 18 hours, or greater than 20 hours. In yet other embodiments, the drug plasma level achieved using a single dose may be greater than 5 ng/ml, greater than 10 ng/ml, greater than 15 ng/ml, greater than 20 ng/ml, greater than 30 ng/ml, greater than 40 ng/ml, greater than 50 ng/ml for a period of 4, 8, 10, 12, 14, 16, 18 or 20 hours.

The maximum plasma concentration of drug may be reached at a time following administration from between 0.1 hr to about 24 hr, or from about 0.25 hr to 10 hr, or from about 0.25 hr to 8 hr, or from about 0.5 hr to 6 hr, or from about 0.5 hr to 4 hr, or from about 0.5 hr to 2 hr, or from about 0.5 hr to 1 hr. The time to maximum plasma concentration may be adjusted by adjusting various components of the ODDS as taught herein. Altering components alters viscosity or other rheological characteristics of the formulation and concomitantly alters rate of drug release (discussed in detail below). The rate of reduction of plasma drug concentration over time may also be adjusted by varying components of the ODDS. Any desired release profile may be achieved by altering components as described herein.

In some embodiments, the release profile of drug over the release period is preferably approximately steady over time, sufficient to provide a therapeutic dose over the release period, and preferably shows a decreased burst effect when compared to a standard tablet formulation. The ODDS of the invention can release drug at an approximately steady rate over a period of at least 24 hours. The release rate is particularly steady from about 1 hr to greater than 24 hrs. This is in contrast to a commercial tablet formulation that provides substantial drug release during the first 5 hr period. The dosage form using the ODDS of the invention provides a long term in vitro release with less than 40% of drug released within 24 hours, whereas the commercial dosage form provides nearly 100% release in 24 hours. The time to 90% release of drug may be varied by varying the formulation and other device components and may be as little as 4 hours, 6 hours, 8 hours, 10, hours, 12 hours, 16 hours or 20 hours, or up to about 24 hours.

The rate of drug release from the dosage form may be varied depending on the drug used and dosage required. For many drugs, they may range from about 0.01 to 500 mg/hr, from 0.5 to 250 mg/hr, 0.75 to 100 mg/hr, 1.0 to 100 mg/hr, 2.0 to 100 mg/hr, 5 to 100 mg/hr, 10 to 100 mg/hr, 10 to 80 mg/hr, 20 to 50 mg/hr, or about 20 to 40 mg/hr.

Dosage regimens for the drug may be determined by the physician in accordance with standard practices. Once per day or twice per day (BID) dosing may be used to maintain a sufficient clinical effect, e.g., to maintain pain relief.

When the medication is an anti-pain medication, an important advantage of the dosage forms disclosed herein is that they have abuse-deterrent characteristics and/or reduced risk of diversion. The dosage form, and the formulation contained therein can be made not susceptible to crushing, powdering or extraction using ethanol or water. For example, HVLCM, if used, is a viscous liquid, and so formulations containing HVLCMs avoid the possibility of crushing for the purpose of inhalation. Additionally, the formulation of the invention has the characteristic of being resistant to drug extraction using ethanol or water, when compared to a tablet formulation of a drug.

Some embodiments of the ODDS disclosed herein of the invention can be used for delivery of a medication that comprise sucrose acetate isobutyrate (SAIB) as a HVLCM carrier material. SAIB is a non-polymeric highly viscous liquid at temperatures ranging from −80° C. to over 100° C., it is a fully esterified sucrose derivative, at a nominal ratio of six isobutyrates to two acetates. It is manufactured by Eastman Chemical Company as a mixed ester, and the resulting mixture does not crystallize but exists as a very viscous liquid. It is a hydrophobic, non-crystalline, low molecular weight molecule that is water insoluble and has a viscosity that varies with temperature. For example, pure SAIB exhibits the viscosity of approximately 2 million centipoise (cP) at room temperature and approximately 600 cP at 80° C. SAIB has unique solution-viscosity relationship in that the SAIB solutions in a number of organic solvents is significantly lower than these viscosity values for the pure SAIB and therefore the SAIB-organic solvent solutions render themselves capable of processing using conventional equipment such as mixers, or liquid pumps. SAIB also has applications in drug formulation and delivery, for example as described in U.S. Pat. Nos. 5,747,058, 5,968,542, 6,413,536, 6,498,153, all incorporated by reference herein. In the present invention, SAIB may be used as the HVLCM and may be present in quantities that vary significantly. For example, quantities of at least about 50, 60, 70, 80, 90, 95, 97, 98, 99, 99.5 or 99.9 wt % can be used. Various formulations containing SAIB are discussed in the examples.

Some embodiments of the ODDS as disclosed allow the gingival delivery of compounds, such as proteins, that would not normally be considered effectively orally administrable because administration in conventional oral compositions would likely result in the breakdown of the active agent by stomach acids or enzymes.

Some embodiments of the invention relate to opioid dosage forms suitable for gingival delivery, including those that provide desirable drug release kinetics and/or limit the likelihood that diversion of the opioids in the dosage forms could occur by patients or others. Suitable opioid compounds deliverable according to the invention include, for example, those generally used as pain relievers, narcotics and/or anesthetics, and include alfentanil, allylprodine, alphaprodine, anileridine, apomorphine, apocodeine, benzylmorphine, bezitramide, buprenorphine, butorphanol, clonitazene, codeine, cyclazocine, cyclorphen, cyprenorphine, desomorphine, dextromoramide, dezocine, diampromide, dihydrocodeine, dihydromorphine, dimenoxadol, dimepheptanol, dimethylthiambutene, dioxyaphetyl butyrate, dipipanone, eptazocine, ethoheptazine, ethylmethylthiambutene, ethylmorphine, etonitazene, fentanyl, heroin, hydrocodone, hydroxymethylmorphinan, hydromorphone, hydroxypethidine, isomethadone, ketobemidone, levallorphan, levorphanol, levophenacylmorphan, lofentanil, meperidine, meptazinol, metazocine, methadone, methylmorphine, metopon, morphine, myrophine, nalbuphine, narceine, nicomorphine, norlevorphanol, normethadone, nalorphine, normorphine, norpipanone, ohmefentanyl, opium, oxycodone, oxymorphone, papaveretum, pentazocine, phenadoxone, phenomorphan, phenazocine, phenoperidine, pholcodine, piminodine, piritramide, propheptazine, promedol, profadol, properidine, propiram, propoxyphene, remifentanyl, sufentanyl, tramadol, tilidine, naltrexone, naloxone, nalmefene, methylnaltrexone, naloxone methiodide, nalorphine, naloxonazine, nalide, nalmexone, nalbuphine, nalorphinedinicotinate, naltrindole (NTI), naltrindoleisothiocyanate, (NTII), naltriben (NTB), nor-binaltorphimine (nor-BNI), beta-funaltrexamine (b-FNA), BNTX, cyprodime, ICI-174,864, LY117413, MR2266, etorphine, DAMGO, CTOP, diprenorphine, naloxone benzoylhydrazone, bremazocine, ethylketocyclazocine, U50,488, U69,593, spiradoline, DPDPE, [D-Ala2,G1u4] deltorphin, DSLET, Met-enkephalin, Leu-enkephalin, 0-endorphin, dynorphin A, dynorphin B, a-neoendorphin, or an opioid having the same pentacyclic nucleus as nalmefene, naltrexone, buprenorphine, levorphanol, meptazinol, pentazocine, dezocine, or their pharmacologically effective esters or salts.

In other embodiments of the invention, a HVLCM, if used, can be used as the continuous phase in a dispersion of particulate biologically active agent. For example, SAIB, which is extremely viscous, can be used to suspend particles of lyophilized protein, microparticles, microspheres, or microcapsules of drugs, for example, biologically active agents, to produce suspension formulations. Concentrations of the active agent in the suspension formulation are analogous to those disclosed above. The resulting suspension formulation has excellent storage stability.

The dosage form of a medication of ODDS of the invention may comprise one or more drugs. The amount of drug(s) and percentages of components in the formulation may vary. Typical average amounts may vary substantially. For many drugs, they may range from about 0.1 mg to 1000 mg, or from about 1 mg to 500 mg, or from about 2 mg to 250 mg, or from about 2 mg to 250 mg, or from about 2 mg to 150 mg, or from about 5 mg to 100 mg, or from about 5 mg to 50 mg. The precise amount of drug desired can be determined by routine methods well known to pharmacological arts, and will depend on the type of drug, and the pharmacokinetics and pharmacodynamics of that drug.

If used, the percent weight of HVLCMs may vary depending on the characteristics of the dosage form desired, and may be for example include from about 30%, 40%, 50%, 60%, 70%, 80%, 90%, 95%, 99%, 99.5%, to about 100%. Exemplary formulations disclosed herein contain 99%, 71%, 70%, 65%, 63%, 61.6%, 59%, 50%, 40%, 30%, 20% or even lesser amounts of SAIB. Variation in SAIB content may be made to alter viscosity or other rheological properties of the formulation and to alter the rate at which drug is delivered. Using the information presented here, ones skilled in the art could alter the SAIB content of the formulation to suit various drugs of differing hydrophobicity or hydrophilicity, and determine the optimum rate of drug release from the formulation.

The dosage form of a medication of ODDS of the invention may comprise one or more solvents. The percent weight of solvent(s) (such as EL) may vary depending on the characteristics of the dosage form desired, and may be for example from about 0% to about 60%, or from about 20% to about 50%, or from about 25% to about 48%. Exemplary formulations disclosed herein include those with 48%, 45%, 36.3%, 31.4%, 29.5%, 29%, 27%, and 23% EL. Again, using the information presented herein, ones skilled in the art could adjust the percent of solvent and determine the optimum amount required for delivery of a particular drug. More than one solvent can be used in a SAIB formulation.

The dosage form of a medication of ODDS of the invention may comprise one or more rheology modifiers. The percent weight of rheology modifier(s) may vary depending on the characteristics of the dosage form desired, and may be for example vary from about 0.1% to about 10%, or from about 0.5% to about 5%, or from about 1% to about 4%. Exemplary formulations disclosed herein include those with 3.5%, 3%, and 1%, and 0%, IM. Using the information presented herein, ones skilled in the art could adjust the percent of formulation viscosity or other rheology modifier and determine the optimum amount required for delivery of a particular drug. More than one rheology modifier can be used in a SAIB formulation.

The percent weight of network former(s) may vary depending on the characteristics of the dosage form desired, and may be for example from about 0% to about 20%, or from about 0.1% to about 10%, or from about 0.5% to about 9%, or from about 1% to about 8.6%. Exemplary formulations disclosed herein include those with 8.6%, 7.8%, 5%, 4.5%, 3%, 2.1%, 2%, 1%, 0.5% and 0% CAB. Different types of CAB (e.g., CAB 381-20, CAB 381-2, and CAB 171-15) may be used to affect desired drug release characteristics. Again, using the information presented herein, ones skilled in the art could adjust the percent of the network former and determine the optimum amount required for delivery of a particular drug. More than one network former can be used in a SAIB formulation.

The formulations of the invention may use network formers such as cellulose acetate butyrate of varying acetyl and butyryl content such as CAB 381-20, CAB 381-2 and CAB 171-15. CAB allows the in-situ formation of a micro-network within the SAIB-drug formulation. Although not wishing to be bound by theory, it appears that the mechanism of micro-network formation appears in part to be due to phase inversion (e.g., change in Tg) of network formers. That is to say, when SAIB formulations containing the CAB type of network former (for example CAB 381-20) are exposed to or immersed in aqueous environments, previously dissolved network formers in SAIB formulations will precipitate as a result of migration of water and other biologically available fluid components, which will result in polymer precipitation process and yield polymeric networks within the ODDS. The formation of micro-network will begin at the surface of the formulation mass and the polymeric network will gradually propagate toward the center of the formulation mass, resulting in a progressive increase in SAIB formulation viscosity in situ.

In a particular embodiment, the invention provides a gingival dosage form comprising a formulation contained within a biodegradable capsule, wherein the formulation comprises a drug, a HVLCM, a rheology modifier, a network former and a solvent, and wherein the capsule is made of a substance that degrades when exposed to conditions present in the gastro-intestinal tract of a mammal. In preferred embodiments, the HVLCM can be SAIB, and the capsule can be made from gelatin or synthetic polymers. In particular embodiments the drug may be an opioid such as oxycodone. The drug-release kinetics of dosage forms incorporating various formulations can be seen to be both unexpected and favorable for delivery of drugs such as oxycodone.

Other Formulations

The medication of the ODDS of the present invention are prepared as other formulations with pharmaceutically acceptable carriers. Preferred are those carriers that will protect the active compound against rapid elimination from the body, such as a controlled release formulation, including implants and microencapsulated delivery systems. Biodegradable, biocompatable polymers can be used, such as polyanhydrides, polyglycolic acid, collagen, and polylactic acid. Methods for preparation of such formulations can be readily performed by one skilled in the art.

Liposomal suspensions (including liposomes targeted to infected cells with monoclonal antibodies to viral antigens) are also preferred as pharmaceutically acceptable carriers. Methods for encapsulation or incorporation of compounds into liposomes are described by Cozzani, I.; Joni, G.; Bertoloni, G.; Milanesi, C.; Sicuro, T. Chem. Biol. Interact. 53, 131-143 (1985) and by Joni, G.; Tomio, L.; Reddi, E.; Rossi, E. Br. J. Cancer 48, 307-309 (1983). These may also be prepared according to methods known to those skilled in the art, for example, as described in U.S. Pat. No. 4,522,811 (which is incorporated herein by reference in its entirety). For example, liposome formulations may be prepared by dissolving appropriate lipid(s) (such as stearoylphosphatidyl ethanolamine, stearoylphosphatidyl choline, arachadoylphosphatidyl choline, and cholesterol) in an inorganic solvent that is then evaporated, leaving behind a thin film of dried lipid on the surface of the container. An aqueous solution of the active compound is then introduced into the container. The container is then swirled by hand to free lipid material from the sides of the container and to disperse lipid aggregates, thereby forming the liposomal suspension.

Other methods for encapsulating compounds within liposomes and targeting areas of the body are described by Sicuro, T.; Scarcelli, V.; Vigna, M. F.; Cozzani, I. Med. Biol. Environ. 15(1), 67-70 (1987) and Joni, G.; Reddi, E.; Cozzani, I.; Tomio, L. Br. J. Cancer, 53(5), 615-21 (1986).

The medication of the ODDS disclosed formed by combining the compounds of this invention and the pharmaceutically acceptable carriers, vehicles or diluents are then readily administered in a variety of dosage forms such as powders, lozenges, syrups, pastes, suspensions and the like. These medication of the ODDS disclosed can, if desired, contain additional ingredients such as flavorings, binders, excipients and the like according to a specific dosage form.

In some embodiments, the medication of the ODDS disclosed provided herein can also be used with another pharmaceutically active agent effective for a disease such as neuro disorders, cardiovascular disorders, tumors, AIDS, depression, and/or type-1 and type-2 diabetes. Such additional agents can be, for example, antiviral agent, antibiotics, anti-depression agent, anti-cancer agents, immunosuppressant, anti-fungal, and a combination thereof.

In some embodiments, the medication of the ODDS disclosed described herein can be formulated alone or together with the other agent in a single dosage form or in a separate dosage form. Methods of preparing various pharmaceutical formulations with a certain amount of active ingredient are known, or will be apparent in light of this disclosure, to those skilled in this art. For examples of methods of preparing pharmaceutical formulations, see Remington's Pharmaceutical Sciences, Mack Publishing Company, Easton, Pa., 19th Edition (1995).

In one embodiment, the substrate may include a carrier that is in the form of a flowable gel. The gel may be selected so as to be injectable periodontically, such as via a syringe at the site of periodontal disease. The gel may be a chemical gel which may be a chemical gel formed by primary bonds, and controlled by pH, ionic groups, and/or solvent concentration. The gel may also be a physical gel which may be formed by secondary bonds and controlled by temperature and viscosity. Examples of gels include, but are not limited to, pluronics, gelatin, hyaluronan, collagen, polylactide-polyethylene glycol solutions and conjugates, chitosan, chitosan & b-glycerophosphate (BST-gel), alginates, agarose, hydroxypropyl cellulose, methyl cellulose, polyethylene oxide, polylactides/glycolides in N-methyl-2-pyrrolidone. See for example, Anatomical Record (2001), 263(4), 342-349, the teachings of which are incorporated herein by reference.

In one embodiment, the carrier may be photopolymerizable, such as by electromagnetic radiation with wavelength of at least about 250 nm. Example of photopolymerizable polymers include polyethylene (PEG) acrylate derivatives, PEG methacrylate derivatives, propylene fumarate-co-ethylene glycol, polyvinyl alcohol derivatives, PEG-co-poly(-hydroxy acid) diacrylatemacromers, and modified polysaccharides such as hyaluronic acid derivatives and dextran methacrylate. See for example, U.S. Pat. No. 5,410,016, herein incorporated by reference.

In one embodiment, the substrate may include a carrier that is temperature sensitive. Examples include carriers made from N-isopropylacrylamide (NiPAM), or modified NiPAM with lowered lower critical solution temperature (LCST) and enhanced peptide (e.g. insulin) binding by incorporation of ethyl methacrylate and N-acryloxysuccinimide; or alkyl methacrylates such as butylmethacrylate, hexylmethacrylate and dodecylmethacrylate (PCT Int. Appl. WO/2001070288; U.S. Pat. No. 5,124,151, the teachings of which are incorporated herein by reference).

In one embodiment, the carrier may include surfactants to promote stability and/or distribution of a pharmaceutically active agent within the carrier materials such as, but not limited to polyoxyester (e.g. polysorbate 80, polysorbate 20 or Pluronic F-68).

In one embodiment, the carrier may include buffering agents such as, but not limited to glycine, glutamic acid hydrochloride, sodium chloride, guanidine, heparin, glutamic acid hydrochloride, acetic acid, succinic acid, polysorbate, dextran sulfate, sucrose, and amino acids. See for example, U.S. Pat. No. 5,385,887, herein incorporated by reference. In one embodiment, the carrier may include a combination of materials such as those listed above.

Tooth Structure Medication

In some embodiments, the tray can be configured to deliver medication for the structure of the tooth (e.g., the enamel) (“tooth structure medication”). In these embodiments, such tooth structure medication is included in the part of the tray apart from the negative groove. For instance, the tooth structure medication can be included in the cavity or walls of cavity. The tooth structure medication can be, for example, a higher concentration hydrogen peroxide (as compared with antibacterial concentration of hydrogen peroxide used and included in the groove of a tray) or other whitening agent, a remineralizing medicine for the enamel, or a combination thereof. During the course of teeth whitening procedures, the enamel becomes slightly damaged (micro pores begin to form). For example, using a remineralizing paste can help to restore the enamel.

Tooth remineralizing agents are known in the art. Examples of such agents include, but are not limited to, sodium calcium phosphosilicate (bioactive glass) ceramic material which can provide calcium, sodium, and phosphate ions to form a hydroxyl carbonate apatite (HCA) was introduced as a remineralizing agent as it can attach to the tooth surface and release ions for remineralization. Remineralization of white-spot lesions and carious lesions may be possible with a variety of currently available agents containing fluoride, bioavailable calcium and phosphate, and casein phosphopeptide in-amorphous calcium phosphate, self-assembling peptide, and other tooth remineralizing agents. (see, e.g., Naveena P, Nagarathana C, Sakunthala BK (2014) Remineralizing Agent—Then and Now—An Update. Dentistry 4: 256).

### EXAMPLES

### Example 1

### Fabrication of an Oral Drug Delivery System (“ODDS”)

Examples of trays of the disclosed ODDS were formed according to an embodiment of method disclosed herein as described below:
A female impression of the patient's teeth and gums that support the teeth from a suitable hardenable material or a digitized scan of the same is made.
Digitized male models of the patient's teeth and gums are made.
The digitized male models are programmed to incorporate one or more raised ridges along the intersection of the tooth and gum line.
The digitized male models are also programmed to incorporate one or more depressed grooves along the intersection of the tooth and gum line.
The programmed digitized male models are 3D printed.
A set of polyethylene-based or other moldable resilient material is molded over the programmed digitized male models to create the oral drug delivery system.
Medication is then applied into the reservoir created by the raised ridges on the digitized male models.
The patient fits the medicine delivery system onto the patient's teeth.
The raised ridge from the depressed grooves on the digitized male models creates a positive pressure and a seal, thereby minimizing leakage of medicine.
The reservoir holds and delivers the medicine through the gum line.

FIG. 1 shows a buccal view of tray 1 of the ODDS. On its teeth touching side, 2 is a negative groove, and 3 is a raised ridge, on its side away from teeth/gum, 2a is a raised ridge, and the negative groove 2 can be filled with a medication to form a drug reservoir 2c (not shown).

FIG. 1 shows a buccal view of tray 1 of an embodiment of the ODDS of invention. On its teeth facing side, 2 is a negative groove, and 3 is a raised ridge, on its side away from teeth/gum, 2a is a raised ridge, and the negative groove 2 can be filled with a medication to form a drug reservoir 2c (not shown).

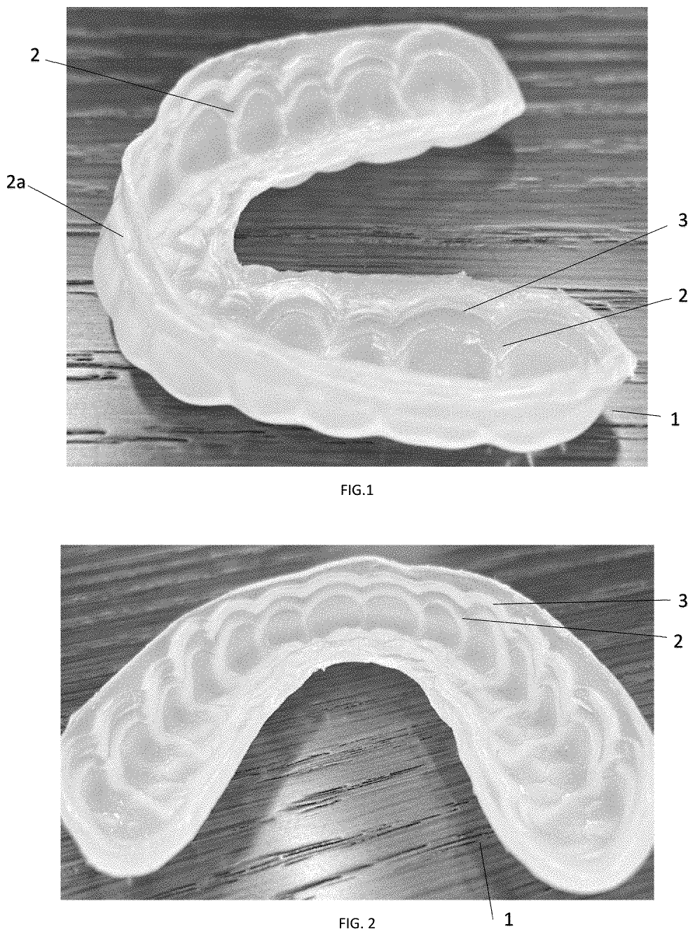

FIG. 2 is a gingival view of tray 1 of an embodiment of the ODDS of invention. On its teeth facing side, 2 is a negative groove, and 3 is a raised ridge, and the negative groove 2 can be filled with a medication to form a drug reservoir 2c (not shown).

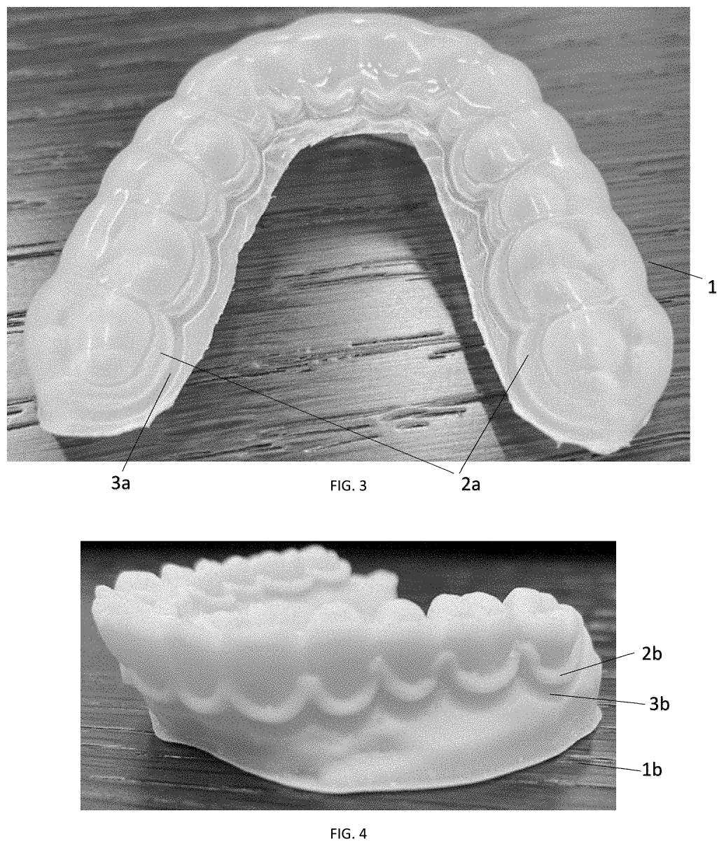

FIG. 3 is an occlusal view of tray 1 of an embodiment of the ODDS of invention. 2a is a raised ridge, which corresponds to a negative groove 2 (not shown) on its tooth facing side, and 3a is a negative groove, which corresponds to a raised ridge 3 (not shown) on its tooth facing side.

FIG. 4 is a mold 1b for forming tray lof an embodiment of the ODDS of invention. 2b is a raised ridge for forming a negative groove on tray 1 (not shown), and 3b is a negative groove for forming a raised ridge 3 on tray 1 (not shown).

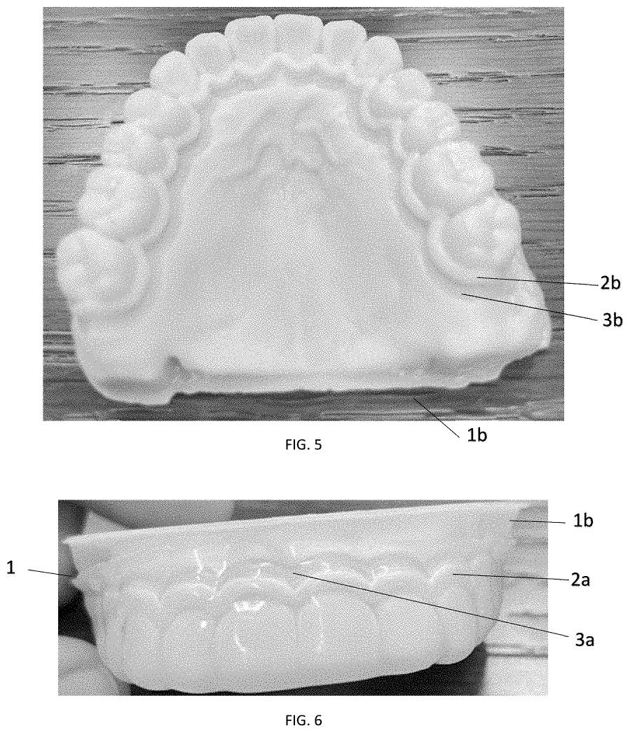

FIG. 5 is an occlusal view of mold 1b for forming tray lof an embodiment of the ODDS of invention. 2b is a raised ridge for forming a negative groove on tray 1 (not shown), and 3b is a negative groove for forming a raised ridge 3 on tray 1 (not shown).

FIG. 6 shows a tray 1 formed on a mold lbof an embodiment of the ODDS of invention. 2a is a raised ridge, which corresponds to a negative groove 2 (not shown) on the tooth facing side of tray 1 (not shown); 3a is a negative groove, which corresponds to a raised ridge 3 (not shown) on the tooth facing side of tray 1 (not shown).

While the present invention has been described in terms of preferred embodiments, it will be appreciated by one of ordinary skill that the spirit and scope of the invention is not limited to those embodiments, but extend to the various modifications and equivalents as defined in the appended claims.

We claim:

1. An oral drug delivery system (“ODDS”), comprising
a tray fitted to at least some or all teeth of a patient's upper or lower arch for the application of medications to the patient in need of a medication, the tray being of resilient elastomeric material molded to substantially conform to the teeth and gum tissue, the tray having at least one cavity formed to conform to the teeth,
one or more raised ridges surrounding the cavity corresponding to a location along the patient's gum line,
one or more negative grooves configured to receive a medication by the raised ridges, the negative grooves surrounding the cavity corresponding to an intersection of the teeth and gum line,
wherein the raised ridge creates a positive pressure on the gum and a seal, thereby minimizing leakage of medicine, and
wherein, as compared with the one or more negative grooves, the one or more raised ridges are further away from the intersection of the teeth and gum line.

2. The ODDS according to claim 1, wherein the one or more negative grooves are filled with a medication forming a drug reservoir.

3. The ODDS according to claim 1, wherein the at least one cavity apart from the negative grooves is filled with a tooth structure medication.

4. The ODDS according to claim 3, wherein the tooth structure medication selected from one or more of a tooth whitening agent, tooth remineralizing agents, and a combination thereof.

5. The ODDS according to claim 2, wherein the medication comprises a pharmaceutically active agent selected from the group consisting of a small molecule drug, a peptide or polypeptide drug and a combination thereof.

6. The ODDS according to claim 5, wherein the pharmaceutically active agent is selected from the group consisting of an antimicrobial agent, an anti-depressant, an anti-neurodegenerative drug, an anti-inflammatory agent, or an anti-pain drug.

7. The ODDS according to claim 5, wherein the medication further comprises a pharmaceutically acceptable carrier.

8. The ODDS according to claim 2, wherein the medication is in a solid formulation or in a solid formulation.

9. A method of forming an oral drug delivery system (“ODDS”), comprising
forming a female impression of a patient's teeth and gums that support the teeth from a suitable hardenable material or performing a digitized scan of a patient's teeth and gums that support the teeth,
forming a digitized male models of the patient's teeth and gums,
programming the digitized male model to incorporate one or more raised ridges along an intersection of the teeth and gum line and one or more depressed grooves by the raised ridges along the intersection of the teeth and gum line, the raised ridges being closer to the intersection of the teeth and gum line as compared with that of the one or more depressed grooves,
forming a male model from the digitized male model,
forming a tray by molding a resilient material over the male model, thereby generating the ODDS having the tray,
wherein the tray is according to claim 1.

10. The method according to claim 9, wherein forming a male model from the digitized male model is by 3D printing.

11. The method according to claim 9, further comprising filling a medication to the one or more negative grooves to form a drug reservoir.

12. The method according to claim 9, further comprising filling the at least one cavity apart from the one or more grooves with a tooth structure medication.

13. The method according to claim 12, wherein the tooth structure medication is selected from the group consisting of a tooth whitening agent, tooth remineralizing agents, and a combination thereof.

14. The method according to claim 11, wherein the medication comprises a pharmaceutically active agent selected from the group consisting of a small molecule drug, a peptide or polypeptide drug and a combination thereof.

15. The method according to claim 14, wherein the pharmaceutically active agent is an antimicrobial agent, an anti-depressant, an anti-neurodegenerative drug, an anti-inflammatory agent, or an anti-pain drug.

16. The method according to claim 12, wherein the medication is in a solid formulation or in a solid formulation.

17. A method of treating a medical condition in a patient, comprising:
determining a presence of a medical condition in the patient,
forming an oral drug delivery system (“ODDS”), the ODDS comprising
a tray fitted to at least some or all teeth of a patient's upper or lower arch for the application of medications to the patient in need of a medication, the tray being of resilient elastomeric material molded to substantially conform to the teeth and gum tissue, the tray having at least one cavity formed to conform to the teeth;
one or more raised ridges surrounding the cavity corresponding to a location along the patient's gum line;
one or more negative grooves as a drug reservoir filled with a medication by the raised ridges, the negative grooves surrounding the cavity corresponding to an intersection of the teeth and gum line; and
optionally a tooth structure medication included in the cavity apart from the negative grooves,
wherein the raised ridge creates a positive pressure on the gum and a seal, thereby minimizing leakage of medicine; and
wherein, as compared with the one or more negative grooves, the one or more raised ridges are further away from the intersection of the teeth and gum line, and
applying the ODDS to the patient.

18. The method according to claim 17, wherein the medication comprises a pharmaceutically active agent that is a small molecule drug or a peptide or polypeptide drug.

19. The method according to claim 17, wherein the medication comprises a pharmaceutically active agent that is an antimicrobial agent, an anti-depressant, an anti-neurodegenerative drug, an anti-inflammatory agent, or an anti-pain drug.

20. The method according to claim 17, wherein the medication further comprises a pharmaceutically acceptable carrier.

21. The method according to claim 20, wherein the medication is in a solid formulation or in a solid formulation.

22. The method according to claim 17, wherein the medical condition is a periodontal disease.

23. The method according to claim 17, wherein the tooth structure medication is selected from the group consisting of a tooth whitening agent, tooth remineralizing agents, and a combination thereof.
